(19) United States
(12) Patent Application Publication
(10) Pub. No.: US 2009/0048493 A1
(43) Pub. Date: Feb. 19, 2009

(54) Health and Entertainment Device for Collecting, Converting, Displaying and Communicating Data

(76) Inventors: Terry L. James, Fairview, TX (US); Grant K. James, Fairview, TX (US)

Correspondence Address: Patent Capital Group, 6119 McCommas Blvd, Dallas, TX, 75214, US

(21) Appl. No.: 11/840,357

(22) Filed: Aug. 17, 2007

(51) Int. Cl. A63F 9/24; A61B 5/00

(52) U.S. Cl. 600/300; 463/1

(57) ABSTRACT

A method for integrating and managing data is provided that includes interacting with an end user via a health and entertainment device, the device including: an accelerometer that is operable to track activity data by the user and a music module operable to play music for the user. The device further includes a memory and a processor operable to convert data from the accelerometer into one or more metrics based on data received from the memory.

### TECHNICAL FIELD OF THE INVENTION

This invention relates in general to a health and entertainment device and, more particularly, to a health and entertainment device for collecting, converting, displaying, and communicating data.

### BACKGROUND OF THE INVENTION

Activity monitors seem almost as ubiquitous today as MP3 players. Together, these devices can serve to inform an end user of his performance data and to entertain/motivate the same user in his workouts. However, their popularity has revealed some of their limitations.

Turning first to the importance of the first component in this equation, activity monitors, these devices are invaluable in a society fraught with obesity issues. Obesity increases the risk of many diseases and health conditions, including diabetes, asthma, hypertension, gallbladder disease, high cholesterol, osteoarthritis, and heart disease. Thirty percent of adults in the United States, who are twenty years or older, are obese. Unfortunately, the same obesity-related diseases and health conditions are becoming more common in younger people as well. The percentage of young people who are overweight has more than tripled since 1980. The percentage of obese children and adolescents is at an all time high. Inactivity and sedentary behavior are among the biggest contributors to the increase of obesity in children, adolescents, and adults.

The United States Surgeon General recommends moderate physical activity for children once a day for at least sixty minutes. Moderate and regular physical activity is good for a person's overall health. Physical activity decreases the risk for cancers, diabetes, high blood pressure, high cholesterol levels, and arthritis. Physical activity also helps control a person's weight, which also positively affects a person's general well being. Physical activity is any bodily movement that results in an expenditure of calories. Moderate physical activity is enough; physical activity does not have to be strenuous to provide benefits to a person's health.

Current health and entertainment devices do not permit the user the option of selecting multiple metrics to characterize their physical activity. Most monitors are limited to one or two metrics. They typically display distance, calories, or intensity as a single metric of activity. A person is more likely to become physically active if the individual is able to see the immediate results of their own physical activity being displayed in a customized format that features a metric that appeals to them. Additionally, a person is more likely to engage in physical activity and sustain that activity if the person can continually monitor their preferred metric and not have to retrieve that metric on a website at a delayed time. Furthermore, a person is more likely to comply with a doctor's orders when a person can view those doctor's orders and monitor their progress in complying with such orders. Providing an incentive for people to engage in physical activity presents a significant challenge and an immense opportunity for those relegated with the onerous task of controlling the population's health.

Concerning the second component of this equation, MP3 players, these devices are generally dumb devices that only appear to be capable of interfacing with other wearable devices through an RF connection. This certainly limits their applications, their utility, and their promise. A new frontier has emerged: How can these popular devices be combined in a way that does not sacrifice their unique capabilities?

Therefore, coordinating between these devices in an optimal manner such that the end user's experience is enriched and augmented offers a significant challenge to those involved in this enormously popular industry.

### SUMMARY OF THE INVENTION

In accordance with the present invention, a method, a system, and a health and entertainment device for collecting, converting, displaying, and communicating data is provided, which substantially eliminates or reduces the disadvantages and problems associated with previous systems, methods, and devices.

In contrast to other systems, the present invention provides for an effective integration between components: providing a single device that offers a musical or entertainment experience, along with an activity monitor. These components are not physically separated, they are physically integrated. In addition, a display is provided for the device to illustrate real-time data for the end user. Also, the device can be connected to a PC download songs or to connect the device to access a website. Numerous other alternatives are discussed below in greater detail.

More specifically, a method for integrating and managing data is provided that includes interacting with an end user via a health and entertainment device, the device including: an accelerometer that is operable to track activity data by the user and a music module operable to play music for the user. The device further includes a memory and a processor operable to convert data from the accelerometer into one or more metrics based on data received from the memory.

In at least one detailed embodiment of the present invention, the device can interface with a web portal located on one or more servers. The web portal is operable to transmit and receive data with a health and entertainment device. The method also involves operating one or more servers coupled to a communication network providing access to the World Wide Web, such that the network is operable to communicate with one or more health and entertainment devices.

The health and entertainment device may include: i) a skin, such that the skin is replaceable with one or more skins being a different color, material, and texture; ii) an accelerometer, such that the accelerometer is operable to track activity data by a user; iii) a memory, such that the memory stores data manually inputted by the user, data received from the accelerometer, and data received by the web portal; iv) a processor, such that the processor is operable to convert data from the accelerometer into one or more metrics based on data received from the memory; v) a communication port, such that the communication port is operable to transmit data to communication network or computer devices and receive data from communication network or computer device; vi) a display; vii) one or more input buttons; and viii) a mode button, such the mode button selects a mode to be displayed.

The modes may include daily points accumulated, average daily points accumulated for a week, activity zone minutes, daily calories expended, total weekly calories expended, daily distance traveled, total weekly distance traveled, an auxiliary mode, wherein the user manually inputs data, a special event mode, and a clock.

In a more particular embodiment, the health and entertainment device is operable to automatically log the user into the web portal. The user can enter user's height, weight, sex, and age on the web portal, and the user can create one or more goals. The web portal is operable to transmit information including but not limited to the user's goals, height, weight, sex, and age to the health and entertainment device via the communication network. The health and entertainment device is operable to convert the data from the accelerometer into calories expended based on the information received from the web portal. The health and entertainment device is further operable to track and display the user's one or more goals received from the web portal. The health and entertainment device is operable to communicate with a machine (for example, a treadmill) to begin a special event and to end a special event, while health and entertainment device collects data during the special event.

Important technical advantages of certain embodiments of the present invention include the ability to achieve a viable integration between two distinct components: a music element and an accelerometer. In addition, the device can be used in conjunction with smart clothing. Also, the web portal transmits user specific data to the health and entertainment device such that the data displayed is customized for each user.

Other technical advantages of the present invention will be readily apparent to one skilled in the art from the following figures, descriptions, and claims. Moreover, while specific advantages have been enumerated above, various embodiments may include all, some, or none of the enumerated advantages.

### BRIEF DESCRIPTION OF THE DRAWINGS

To provide a more complete understanding of the present invention and features and advantages thereof, reference is made to the following description, taken in conjunction with the accompanying figures, wherein like reference numerals represent like parts, in which:

FIG. 1A is a simplified block diagram that illustrates a communication system in accordance with a particular embodiment of the present invention;

FIG. 1B is a simplified schematic diagram that illustrates a health and entertainment device in accordance with a particular embodiment of the present invention;

FIG. 2A is a simplified schematic diagram that illustrates an example end user and smart clothing being used with the device in accordance with a particular embodiment of the present invention;

FIG. 2B is a simplified block diagram that illustrates the device in accordance with a particular embodiment of the present invention;

FIG. 2C is a simplified block diagram that illustrates how the device can be used with a computer in accordance with a particular embodiment of the present invention;

FIG. 3 is a simplified flowchart that illustrates an example method of the communication system in accordance with an embodiment of the present invention;

FIG. 4 is a simplified flowchart that illustrates an example method of the special event feature of the health and entertainment device in accordance with an embodiment of the present invention;

FIG. 5A is an example of the display of a user's daily points in accordance with an embodiment of the present invention;

FIG. 5B is an example of the display of a user's goal for total weekly calories in accordance with an embodiment of the present invention; and

FIG. 5C is an example of the display of a user's goal for pills consumed in auxiliary mode in accordance with an embodiment of the present invention.

### DETAILED DESCRIPTION OF EXAMPLE EMBODIMENTS

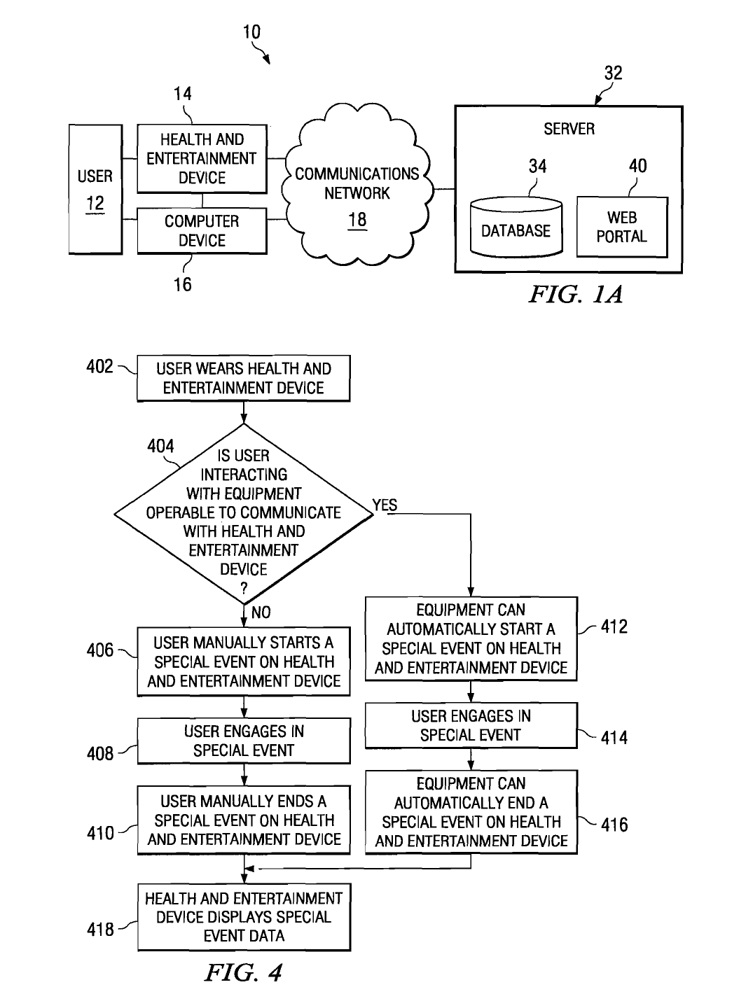

FIG. 1A is a simplified block diagram of a communication system 10 for collecting, displaying, converting, and communicating data. System 10 includes a communication network 18, one or more end users 12, one or more computer devices 16, one or more health and entertainment devices 14, one or more servers 32, one or more databases 34, and a web portal 40. Other architectures and components of system 10, including various architectures and components of server 32, may be used without departing from the scope of this disclosure.

In general, users 12 can wear health and entertainment device 14 to track one or more primary metrics. Users 12 can couple health and entertainment device 14 to one or more computer devices 16, which provide users access to a web portal 40. Health and entertainment device 14 can transmit data to web portal 40.

In accordance with the teachings of the present invention, device 14 offers a significant synergy between two valuable components: an accelerometer and a music-playing component (e.g. an MP3 player). A key ancillary component is the audio feedback for the end user, which can alert the wearer of his intensity, pace, speed, heart-rate (HR), duration, etc. Note that other music players can certainly be included within device 14, as the MP3 offers only one potential protocol or format to be used in conjunction with the present invention.

In addressing these two distinct components, the objective is to integrate them in a seamless fashion. Note that there is a difference between connecting these two instruments and actually achieving an effective integration. For example, current electronic systems may allow an end user to wear an activity monitor in one location (e.g. on a shoe), whereby it could send a message to an MP3 player using a radio-frequency connection. But this activity data is not visible for the end user; it is not providing real-time useful feedback. In order to see what the end user has accomplished, the accelerometer needs to be plugged into a computer (via a conventional USB port or wireless interface). This is truly the only way that the end user can see his results from the previous activity.

In contrast to these shortcomings, the present invention provides for an integration between components: providing a single coherent device. These two components are not physically separated; they are physically integrated, which is critical. In addition, a display is provided for device 14 to illustrate real-time data for the end user. Also, health and entertainment device 14 can easily be connected to a computer to download songs or to facilitate a connection between the device and a website. [Note that the following two related cases, which address aspects of the aforementioned website and unique features of the activity monitor, are hereby incorporated by reference: Accelerometer for Data Collection and Communication, Ser. No. 10/996,500, filed Nov. 23, 2004; System and Method for Implementing an Interactive Online Community Utilizing an Activity Monitor, Ser. No. 11/625,063, filed Jan. 19, 2007.]

In a simple design, device 14 includes an accelerometer, an MP3 player, and either a USB or an RFID (wireless) connection. Health and entertainment device 14 also enables wireless games to be played in real-time. Additionally, and as illustrated in the subsequent FIGURES, device 14 (and its associated connections, wires, etc.) can be embedded into apparel such as: shorts, underwear, bras, shirts, shoes, hats, or any other suitable article of clothing.

Health and entertainment device 14 also allows for end users to upload their workouts (along with music, routines, etc.) to device 14 so that people can choose their own workouts, or complete the workouts of other people and, furthermore, have the workouts show up on the display such that they can be verified, or checked-off as the workout progresses. Simple logging capabilities are easily accommodated by device 14.

Furthermore, these workouts can have custom music or include the same music that was originally designated by the people who designed the workouts. The musical component of device 14 would be able to show what song is being played, or the display could just run off a playlist. Some songs could be pre-selected such that when activities reach a given threshold (high or low), as verified by the accelerometer, a specific song is played.

The inherent wireless connection of device 14 will be able to upload information automatically to the Internet, as device 14 is used anywhere. This could allow, for example, an end user to compete in online games throughout the day, or to challenge other users on a piece of fitness equipment such as a treadmill, stationary bike, etc.

Turning to the website component of device 14, communication system 10 also achieves an effective way for users 12 to view activity data. Web portal 40 is operable for users 12 to input customized data, such that the data is unique to each user 12. Web portal 40 is operable to transmit this customized data to health and entertainment device 14. Health and entertainment device 14 is operable to monitor, calculate, and display user's physical activity in a format selected by user 12. Health and entertainment device 14 can display user's current level of performance, or health and entertainment device can continually update and display user's progress for achieving one or more goals.

System 10 offers advantages to a group of users who seek to monitor physical activity. This is due, at least in part, to health and entertainment device 14, which is capable of displaying several different activity modes. For example, user 12 can select to view the progress towards achieving a goal, the number of calories expended in the past week, the number of pills taken for the day, the distance traveled during a special event, or the number of points accumulated for the day. Details relating to these operations are explained below in conjunction with accompanying FIGURES.

Communication network 18 couples and facilitates wireless or wire-line communication between computer devices 16, health and entertainment devices 14, and servers 32. Communication network 18 may, for example, communicate Internet Protocol (IP) packets, Frame Relay frames, Asynchronous Transfer Mode (ATM) cells, voice, video, data, and other suitable information between network addresses. Communication network 18 may also communicate data via wireless communications, such as by Wireless Application Protocol (WAP) standard protocols, including 802.11, third-generation (3G) protocols (such as W-CDMA or CDMA 2000, for example), Bluetooth, or Global System for Mobile Communications (GSM) protocols, for example. Communication network 18 may include one or more local area networks (LANs), radio access networks (RANs), metropolitan area networks (MANs), wide area networks (WANs), interactive television networks, all or a portion of the global computer network known as the Internet, and/or any other communication system or systems at one or more locations.

Users 12 may include any individual, group, or business entity desiring to use a health and entertainment device 14 or a computer device 16 to access web portal 40. Users 12 may wear health and entertainment devices 14 and couple health and entertainment devices 14 to one or more computer devices 16 to connect to web portal 40. Some users 12 might not wear health and entertainment devices 14, but these users 12 may monitor the activity data of one user 12 or a population of users 12 by accessing and viewing web portal 40. For example, user 12 may be a physical education instructor who uses the computer device 16 to create a curriculum on web portal 40 that utilizes health and entertainment device 14. Users 12 may also include physical education students who couple their health and entertainment devices 14 to computer device 16 to transmit the data from health and entertainment device 14 to web portal 40. Web Portal 40 allows teachers to view the physical activity data of their students and use this information to grade the students according to the curriculum.

In another embodiment, user 12 may be a business entity that utilizes health and entertainment device 14 to attract potential users to the business. Health and entertainment device 14 may provide helpful information to user about the business. Health and entertainment device 14 can grant users 12 access to the business's web portal 40 that serves as an online business center. Multiple forms of communication and information can be passed from the business to users 12 via web portal 40. Health and entertainment device 14 will engage users to visit the business's web portal 40.

In another embodiment, users 12 may include one or more doctors and one or more patients such that doctors can utilize web portal 40 and health and entertainment devices 14 to provide treatment to patients. Patients may wear health and entertainment device 14 and transmit their physical activity data to web portal 40. Doctors can access and monitor their patients' daily and weekly physical activity data. Doctors can also monitor their patients' compliance to consuming fruits, vegetables, and pills and/or monitor/assign specific medical workouts (e.g. cardiac or pulmonary rehabilitations.).

Health and entertainment devices 14 are generally operable to measure body movement of a user, store data, receive data, transmit data, display data, and convert data for a multitude of purposes. For example, health and entertainment devices 14 are operable to continuously communicate with server 32 using wireless communication, such that server 32 and health and entertainment device 14 are continuously updated with real time data. For example, health and entertainment device 14 may measure a primary metric that includes calories, distances, points, life zone minutes, health zone minutes, or sports zone minutes. User 12 can wear health and entertainment device 14 for an entire day or only for an event for a specified period of time.

Memory in health and entertainment device 14 may include volatile or non-volatile memory including, without limitation, magnetic media, optical media, random access memory (RAM), read-only memory (ROM), removable media, or any other suitable local or remote memory component. In general, the memory may store various data including metrics, a user's account information, a user's goals, and a user's weight, height, age, and sex in any suitable format. For example, user's account information may include a unique identification number associated with each user 12. Health and entertainment device 14 is operable to receive data from web portal 40, computer device 16, machine, or any other device.

Health and entertainment device 14 is further operable to transmit data to web portal 40, computer device 16, or any other device. Health and entertainment device 14 may include a graphics card to display streaming video and data stored in memory. Health and entertainment device 14 may include a processor to convert data and utilize algorithms. For example, health and entertainment device 14 may apply an algorithm to measure distance traveled or calories burned by utilizing data transmitted from web portal 40 to the memory of health and entertainment device 14. Health and entertainment device 14 is further operable such that health and entertainment device 14 can be disabled from displaying data, from transmitting data, and from receiving data.

A feature of the present invention includes a subscription model that may include users 12 paying to use web portal 40 and paying to use health and entertainment device 14. For example, server 32 and health and entertainment device 14 are operable for server 32 to disable and/or enable certain functions and modes of health and entertainment device. Server 32 can configure all health and entertainment devices 14 of a group of users 12, such that all health and entertainment devices 14 used by a particular business entity are configured with the same functionality. If user 12 is delinquent in subscription payments, health and entertainment device 14 may be disabled completely and access to web portal 40 may be blocked. Additional details of health and entertainment device 14 are listed below.

Software and/or hardware may reside in health and entertainment device 14 in order to achieve the teachings of collecting data, converting data, displaying data, and communicating data of the present invention. However, due to its flexibility, health and entertainment device 14 may alternatively be equipped with (or include) any suitable component, device, application specific integrated circuit (ASIC), processor, microprocessor, algorithm, read-only memory (ROM) element, random access memory (RAM) element, erasable programmable ROM (EPROM), electrically erasable programmable ROM (EEPROM), field-programmable gate array (FPGA), or any other suitable element or object that is operable to facilitate the operations thereof. Considerable flexibility is provided by the structure of health and entertainment device 14 in the context of communication system 10 and, accordingly, it should be construed as such.

Computer devices 16 may comprise computer systems that include appropriate input devices, output devices, mass storage media, processors, memory, or other components for receiving, processing, storing, and/or communicating information with other components of system 10. As used in this document, the term “computer” is intended to encompass a docking station (although USB connections may obviate the need for a docking station entirely), personal computer, workstation, network computer, wireless data port, wireless telephone, personal digital assistant (PDA), cellular telephone, game console, one or more processors within these or other devices, or any other suitable processing device. It will be understood that any number of computer devices 16 may be coupled to other computer devices 16 or communication network 18. Computer devices 16 are generally operated by users 12 or coupled with health and entertainment devices 14 to access the interactive community.

A particular computer device 16 may comprise a browser application, such as an Internet web browser, for example. Browser application may allow user 12 of computer device 16 to navigate through, or “browse,” various Internet web sites or web pages. Computer device 16 may also comprise one or more graphics applications, such as a FLASH™ application for example, operable to display various types of data received via communication network 18, such as graphics, video, and streaming data (such as video and/or audio), for example.

A particular health and entertainment device 14 can be coupled to computer device 16 such that user 12 can access the web portal 40 without intervention from a third party (for example, a webmaster forwarding information). Health and entertainment device 14 functions as a digital key to web portal 40 so that users instantly access web portal 40 without having to launch an Internet web browser or type in a username or password. The user will be able to instantly interact with web portal 40.

Servers 32 are generally operable to provide an interface between users 12 and web portal 40. One or more servers 32 may be web application servers or simple processors operable to allow users 12 to participate with web portal 40 via the communication network 18 using a standard user interface language such as, for example, the Hypertext Markup Language (HTML). In some embodiments, one or more servers 32 may be physically distributed such that each server 32, or multiple instances of each server 32, may be located in a different physical location geographically remote from each other. In other embodiments, one or more servers 32 may be combined and/or integral to each other. One or more servers 32 may be implemented using a general-purpose personal computer (PC), a Macintosh, a workstation, a UNIX-based computer, a server computer, or any other suitable processing device. Server 32 may include a processor to convert data and utilize algorithms. For example, server 32 may apply an algorithm to convert distance traveled into calories burned by utilizing data from the memory like a user's height, weight, and sex.

In some embodiments, servers 32 are operable to provide security and/or authentication of users 12 or other persons or entities attempting to access web portal 40. For example, servers 32 may essentially provide a firewall for entities attempting to access web portal 40. In addition, servers 32 may be operable to translate one or more data protocols used by web portal 40 with one or more protocols used by applications hosted by one or more computer devices 16.

In particular embodiments, one or more servers 32 are web application servers operable to communicate dynamically updated information to particular computer devices 16 via communication network 18 including the identity of user 12. For example, one or more servers 32 may communicate updated information on web portal 40 to particular computer devices 16 or health and entertainment devices 14 via communication network 18.

Servers 32 further comprise a memory that may be accessed or otherwise utilized by one or more components of interactive community. The memory may take the form of volatile or non-volatile memory including, without limitation, magnetic media, optical media, random access memory (RAM), read-only memory (ROM), removable media, or any other suitable local or remote memory component. In general, the server memory may store various data including a user's account information, a user's goals, a user's activity data, and a population's activity data.

Databases 34 are operable to store various data associated with web portal 40, such as information regarding users 12, computer devices 16, and health and entertainment devices 14. Databases 34 may communicate with servers 32 such that servers 32 may store information, retrieve information, and share information with each other. Databases 34 may provide a backup in the case of outages or other failures of various components of web portal. Other architectures and components of servers 32 may be used without departing from the scope of this disclosure.

Web portal 40 comprises one or more web sites, hardware, and software that provide users of the web with the ability to search for information on the web including information in the web portal 40, documents, media, or other resources coupled to the web. The web sites on web portal 40 may include user's websites and informational websites. Web portal 40 provides a central location for users to get together with each other.

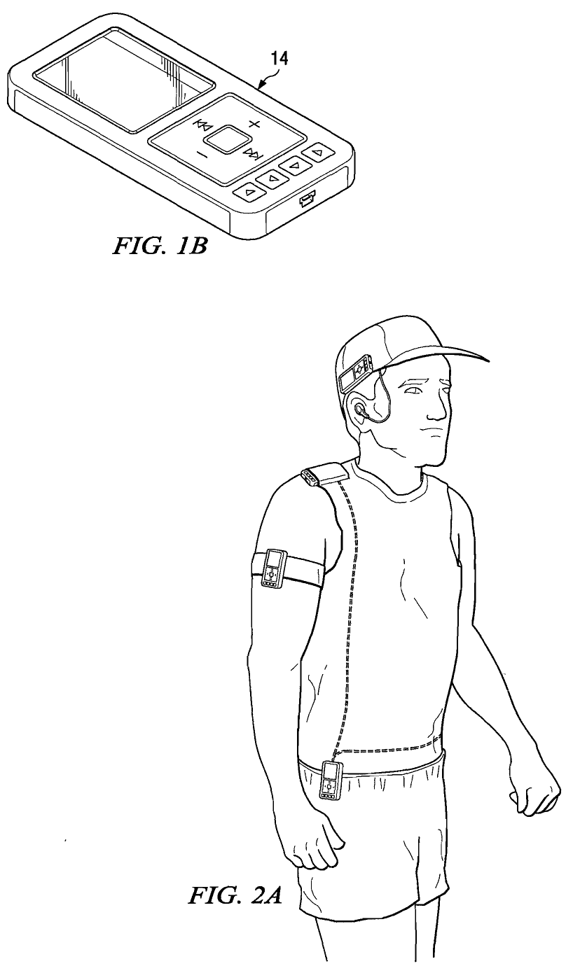

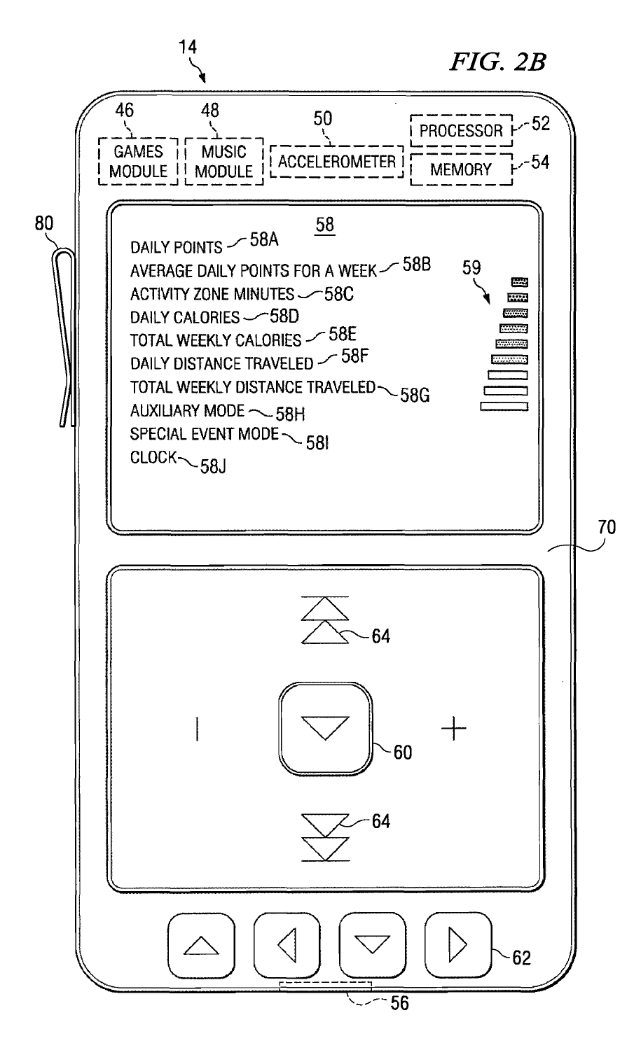

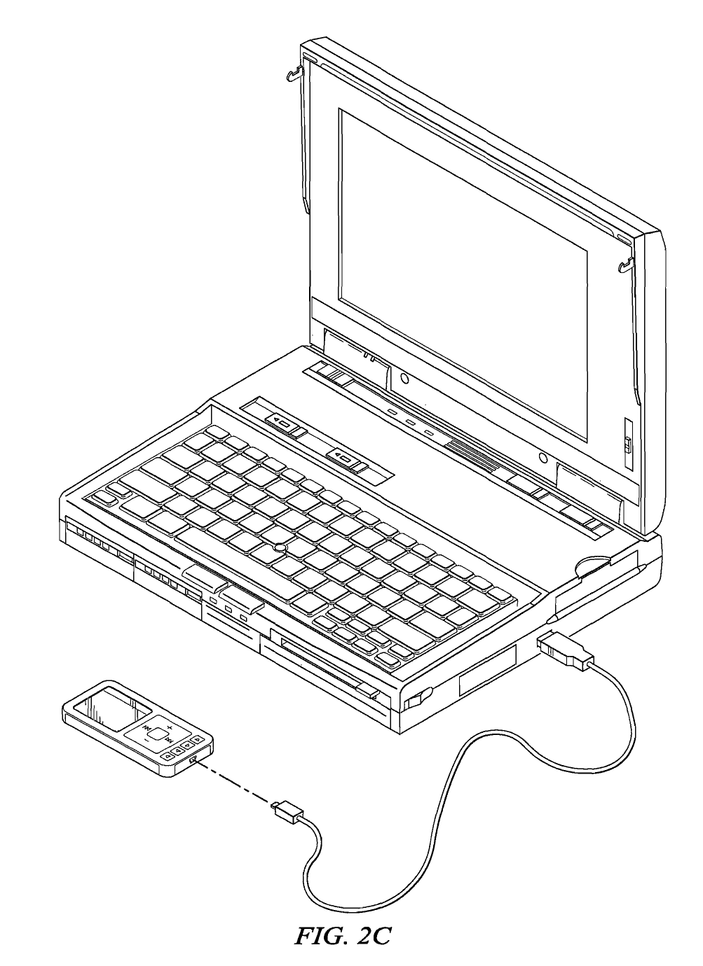

FIG. 1B is a simplified schematic diagram that illustrates health and entertainment device 14 in accordance with a particular embodiment of the present invention, while FIG. 2A is a simplified schematic diagram that illustrates an example end user and smart clothing being used in accordance with a particular embodiment of the present invention. Note that aesthetics are important in the field of wireless technologies. The ‘science’ behind a certain tool (e.g.) is only half of the equation, as there is an increased emphasis on appearance. Just as important, device 14 needs to be lightweight, durable, and wearable: meaning that it should fit against the end user in a snug fashion and be secure during the workout. There are generally predefined locations (or pockets or receptacles) in smart clothing that can receive a given device. These fabricated receptacles of the apparel allow for an easy deposit or attachment for a given device prior to commencing a user's workout.

Device 14 measures movement, provides a pleasant music experience, offers audio feedback, and can easily be clipped to clothing that may or may not be smart clothing, which is generally designed to hold, carry, and receive certain electronic devices. Smart clothing offers the ability to include these devices in a sleek manner. Smart clothing also allows for ready-made connections such that a given device can simply be clipped into the apparel.

The ability to secure device 14 to portions of the body other than the foot is important. As a general premise, trunk movement is more accurate than foot movement in terms of activity surveillance. There appears to be some distortion that occurs through foot volatility: volatility that is not as prominent when a device is mounted on the trunk of the user.

With respect to the audio feedback component of device 14, the actual data may be recorded by the end user, or it may be standardized or selected from a menu. These items may include HR, intensity, duration, speed, distance, how long an end user was exercising in a given sport zone, health zone, etc. In certain embodiments, the audio feedback is triggered via completion of a portion of the workout, or based on the activity of the end user. For example, if activity has increased to a higher level or has spiked, the audio feedback may offer praise, or it may offer a warning. In other scenarios, if the activity level has dropped, then encouragement could be offered. Other triggers for the audio feedback include a certain time that has been reached, or a certain caloric burn that has been achieved. Virtually any parameter that is being measured can be used as a basis for triggering the audio feedback functionality of device 14. Furthermore, the audio feedback may be used in conjunction with playing certain music: for example, to motivate the end user.

FIG. 2B is a simplified block diagram that illustrates health and entertainment device 14 for collecting, displaying, converting, and communicating data in accordance with a particular embodiment of the present invention. Health and entertainment device 14 includes a games module 46, a music module 48, an accelerometer 50, a processor 52, a memory 54, a port 56, a display 58, a mode button 60, a special event button 62, one or more input buttons 64, a skin 70, and a clip 80. Display 58 is operable to display an activity meter 59 and several different modes including daily points 58A, average daily points for a week 58B, activity zone minutes 58C, daily calories 58D, total weekly calories 58E, daily distance traveled 58F, total weekly distance traveled 58G, auxiliary mode 58H, special event mode 58I, and a clock 58J.

Accelerometer 50 is a device that is used to convert an acceleration from gravity or from motion into an electrical signal. The input for accelerometer 50 is generally gravity or motion. Accelerometer 50 can measure acceleration in units of “g's”. One “g” is defined as the earth's gravitational pull on an object or a person. For example, 1g represents the acceleration exerted by the Earth's gravity on an object or person (for example, a cell phone on a desk experiences 1g of acceleration). The acceleration range experienced by a person when walking is between 0.1-2.0 g. Accelerometer 50 measures all user activity by instantaneously tracking the full motion and force (for example, acceleration and deceleration) of user's hips and torso.

Processor 52 controls the operation and administration of health and entertainment device 14 by processing information and signals. Processor 52 includes any suitable hardware, software, or both that operate to control and process signals. Processor 52 may be microprocessors, controllers, or any other suitable computing devices, resources, or combination of hardware, software and/or encoded logic. For example, processor 52 may be used to calculate calories by utilizing data from accelerometer 50 and data from web portal 40.

Music module 48 and games module 46 can include preloaded items, items selected from a menu, or these modules can receive information wirelessly or via a USB connection. In one instance, these items can readily receive downloads from a PC such that music and games can be updated periodically. In addition, these items can be used to exchange music between end users or to play games amongst individuals in real-time. FIG. 2C is a simplified block diagram that illustrates how device 14 can be used with a computer in accordance with such an implementation.

Memory 54 may be accessed or otherwise utilized by health and entertainment device 14. Memory 54 may take the form of volatile or non-volatile memory including, without limitation, magnetic media, optical media, random access memory (RAM), read-only memory (ROM), removable media, or any other suitable local or remote memory component. In general, memory 54 may store various data including data from accelerometer, data from processor, and data from web portal.

Port 56 may communicate information and signals to one or more computer devices 16 and receive information and signals from one or more computer devices 16. Port 56 may also communicate information and signals to communication network 18 and receive information and signals from communication network 18. Port 56 represents any connection, real or virtual, including any suitable hardware and/or software that may allow health and entertainment device 14 to exchange information and signals with communication network 18, one or more computer devices 14, and/or other elements of system 10. For example, port 56 enables health and entertainment device 14 to receive data from web portal 40. Port 56 further enables health and entertainment device 14 to transmit data to web portal 40 including all updated activity data.

Display 58 is operable to display one or more images in one or more formats. Images viewed in display 58 may include daily points 58A, average daily points for a week 58B, activity zone minutes 58C, daily calories 58D, total weekly calories 58E, daily distance traveled 58F, total weekly distance traveled 58G, auxiliary mode 58H, special event mode 58I, a clock 58J, and an activity meter 59.

Daily points 58A can be viewed on display 58. Daily points 58A are the points user 12 has accumulated in one day. Points can be in a format that is easier for user 12 to understand than other data formats. For example, points may be a two-digit number that is easily understood by user 12 to quickly indicate how active user 12 has been during the course of user's 12 daily life routine. Points may be calculated by multiplying the following ratio by 100, wherein the ratio is the amount of user's energy expended while active and the amount of user's energy expended while at rest. Walking for half an hour may result in seven to fourteen points. Running for half an hour may result in fifteen to thirty points. The exact number of points accumulated will depend on the user's activity. The daily points 58A provide user 12 with a simple and straightforward method to quantify and express the total amount of activity that user 12 achieves over a single day. The average daily points for a week 58B allows user 12 to track how consistent user 12 has been active for the past seven days. Web portal 40 or other literature may indicate the amount of daily points 58A users 12 should strive to accumulate to achieve a healthy lifestyle. A younger user may need to accumulate a high number of points to lead a very active lifestyle, while an older user may need to accumulate a lower number of points to lead a very active lifestyle. By displaying a simple format like points, health and entertainment device 14 engages user 12 to stay active until user 12 has accumulated enough points because user 12 does not have to track more complicated metrics.

Activity zone minutes 58C can be viewed on display 58. Activity zones may display life zone minutes, health zone minutes, and sport zone minutes. Life zone minutes may include activity consisting of physical activity experienced in the course of daily living such as walking around the house. Health zone minutes may include walking activity or comparable activity consistent with recommendations from the medical community necessary for a beneficial health effect, i.e., such as walking thirty minutes a day most days of the week at some prescribed level of effort. Sport zone minutes may include running activity or activity with similar physical intensity. Web portal 40 or other literature may indicate the amount of time user 12 should strive to accumulate in the activity zones to achieve a healthy lifestyle. Displaying activity zone minutes 58C engages user 12 to stay active until user 12 has accumulated enough activity zone minutes 58C.

Daily calories expended 58D can be viewed on display 58. Health and entertainment device 14 can calculate an accurate amount of calories expended by user 12 by utilizing user's weight, height, sex, and age. Health and entertainment device 14 receives updated information from web portal 40 every time that health and entertainment device 14 connects to web portal 40 such that user 12 never has to manually input data like height, sex, age, and weight into health and entertainment device 14. For example, health and entertainment device 14 can continuously be connected to web portal 40, such that health and entertainment device 14 continuously received information from web portal 40. The total weekly calories expended 58E can also be viewed on display 58. Web portal 40 or other literature may indicate the amount of calories user 12 should expend to achieve a healthy lifestyle. Displaying the amount of calories expended engages user 12 to stay active until user 12 has expended enough calories.

Daily distance traveled 58F can be viewed on display 58. Health and entertainment device 14 may allow user 12 to set the measurement of distance including feet, miles or kilometers, etc. Total weekly distance 58G traveled can also be viewed on display 58. Web portal 40 or other literature may indicate the amount of distance users 12 should travel to achieve a healthy lifestyle. Displaying the amount of distance traveled engages user 12 to stay active until user 12 has traveled far enough.

Auxiliary mode 58H can be viewed on display 58. In auxiliary mode 58H, user 12 can manually input numbers into health and entertainment device 14. For example, a physician may give user 12 a regimen to take three pills a day or eat five vegetables a day [or other suitable items such as cups of water drunk, grams of protein consumed, a number of times a task was done, etc.]. Physician or user 12 may input this information into web portal 40. Web portal 40 can transmit this information to health and entertainment device 14 such that health and entertainment device 14 can display this information. Health and entertainment device is operable for user 12 to manually input each time user 12 takes a pill or eats a vegetable, such that the auxiliary mode displays the updated information. User 12 may press a button on health and entertainment device 14 for every pill or vegetable. User 12 can connect health and entertainment device 14 to web portal 40, such that auxiliary mode 58H information is automatically transmitted to web portal 40. Physician may monitor web portal 40 to make sure user 12 is in compliance of a regimen (for example, user is taking the number of pills per day and eating the number of vegetables per day). Auxiliary mode 58H enables user 12 to properly track a diet regimen or program. Users 12 may not remember how many pills that they have taken throughout the day, and auxiliary mode 58H enables users 12 to track their personal regimen. Physicians can also monitor their patients to make sure that patients are compliant with the regimen prescribed for them.

Special event mode 58I, which can be viewed on display 58, enables user 12 to begin a special event 58I (e.g. walking on an incline of a treadmill) and to end special event 58I. Additionally, special event mode 58I enables machines, like a treadmill, to begin a special event and to end a special event. For example, a treadmill may send a signal to health and entertainment device 14 to begin a special event when the treadmill is turned on and to end a special event when the treadmill is turned off. The health and entertainment device 14 will track the activity data during the special event 58I time period, such that user 12 can monitor activity of specific events. Alternatively, user 12 can manually press a button for special event 58I to begin at the start of a marathon and manually press a button for special event 58I to end when user 12 crosses the finish line. Special event mode 58I enables users to monitor specific activity events, which engages users 12 to become more active.

Clock 58J can be viewed on display 58. Clock 58J can be the time of day. Clock 58J can also be a stopwatch to monitor the amount of time spent on an activity. Activity meter 59 can be viewed on display 58. Activity meter 59 can be one or more bars such that no bars are displayed while user 12 is stationary, and the number of bars displayed will increase as user's current activity level increases.

Mode button 60 on health and entertainment device 14 enables user 12 to toggle through one or more display modes for user 12 to view. For example, user 12 can press mode button 60 to toggle display 58 from daily points to daily calories expended 58D to special event mode 58I, etc. Special event button 62 on health and entertainment device 14 enables user 12 to begin and to end a special event. One or more input buttons 64 on health and entertainment device 14 enable user 12 to input information like incrementing the counter in auxiliary mode 58H.

Skin 70 encases the outside of health and entertainment device 14. Skin 70 can be removable with one or more skins 70. Skin 70 can have different features including a different color, material, and texture. Clip 80 can attach to the back of health and entertainment device 14. Clip 80 enables user 12 to easily attach health and entertainment device 14 to an article of clothing. Clip 80 can be removable with one or more clips 80. Clip 80 can have different features including a different color, material, and texture. Removable, unique skins 70 and clips 80 allows user 12 to customize the appearance of health and entertainment device 14. Users 12 are more likely to wear health and entertainment device 14 by customizing the look of health and entertainment device 14.

For purposes of teaching and discussion, it is useful to provide some overview as to the way in which the following invention operates. The following foundational information may be viewed as a basis from which the present invention may be properly explained. Such information is offered earnestly for purposes of explanation only and, accordingly, should not be construed in any way to limit the broad scope of the present invention and its potential applications.

Physical inactivity is a major risk factor for a multitude of illnesses. Precise quantification of physical activity is critical in any environment, whether it be in the realm of heightened physical fitness or in situations where some individual is in poor health. Physical activity is especially important in measuring the outcomes in frail, sedentary populations, and in the elderly, because small improvements in physical functioning such as walking and balance may translate into significantly improved higher-order function and life quality. Nonetheless, although daily monitoring of physical activity is of great interest to investigators and clinicians alike, methods to precisely measure this vital dimension of function have only been recently available.

Methods in current use for measuring daily activity include direct observation, self-report questionnaires and diaries, radioisotope techniques (doubly-labeled water measurement of energy expenditure), and heart-rate monitoring. These methods suffer from several problems. Direct observation is both time-consuming and intrusive, and self-report questionnaires and diaries that rely on memory are imprecise (especially in the elderly) and are time-intensive for subjects. Radioisotope methodology is both costly and technologically complex. Heart-rate monitoring is both expensive and imprecise in patients whose heart rates may vary due to medication use and other causes unrelated to physical activity. In addition, neither radioisotope and heart-rate monitoring technologies are capable of providing information on specific patterning of activity.

Accelerometer 50 movement sensors are a practical alternative to other methods, providing a high degree of precision across a wide range of activity levels at a relatively low cost. Single-axis accelerometers measure movement in one plane and have been widely used to study physical activity and energy consumption in healthy young people and the elderly. In addition to having only one plane of measurement, a major disadvantage of some activity devices is inadequate data storage and retrieval technology, which requires study subjects to read and record output from the device. More recently, a new generation of multi-axis activity monitors have been developed that have improved sensitivity and are more suitable for research purposes. (Note that health and entertainment device 14 may be a single plane, dual-plane, tri-plane, or multi-plane device.)

Health and entertainment device 14 is operable to transmit activity zone data 58C to web portal 40. Web portal 40 is operable to monitor energy expenditure in normal, active populations, and to monitor activity in relatively sedentary clinical populations, including nursing home residents, outpatients with multiple sclerosis (MS), and obese children.

Activity measurement of walking would assist greatly in clarifying the role of walking behavior as a marker and perhaps determinant of physical functioning in the elderly and persons with chronic illness. Walking is the activity targeted for improvement in most cardiac and pulmonary rehabilitation programs and other health-maintenance regimens aimed at improving physical functioning, prolonging life, and preventing illness associated with sedentary living. In other scenarios, accelerometer measurements of running sessions or workouts provide an invaluable training tool for the serious athlete. Health and entertainment device 14 can accurately gauge activity associated with vigorous training regimens. In addition, the collected data may serve as a terrific training log for any athlete, as his progress and daily energy expenditures may readily be determined.

When looking at energy utilization, there are generally four types of approaches for measuring energy expended by an individual: i) oxygen consumption; ii) heart-rate monitoring; iii) pedometers; and iv) accelerometers 50. Pedometers are highly inaccurate, heart-rate monitoring measurements are often skewed (for example, due to conditioning, deconditioning, drugs, etcetera), and direct oxygen consumption measurements are time-consuming, cumbersome, and expensive. Hence, accelerometers 50 provide a viable alternative to these flawed devices.

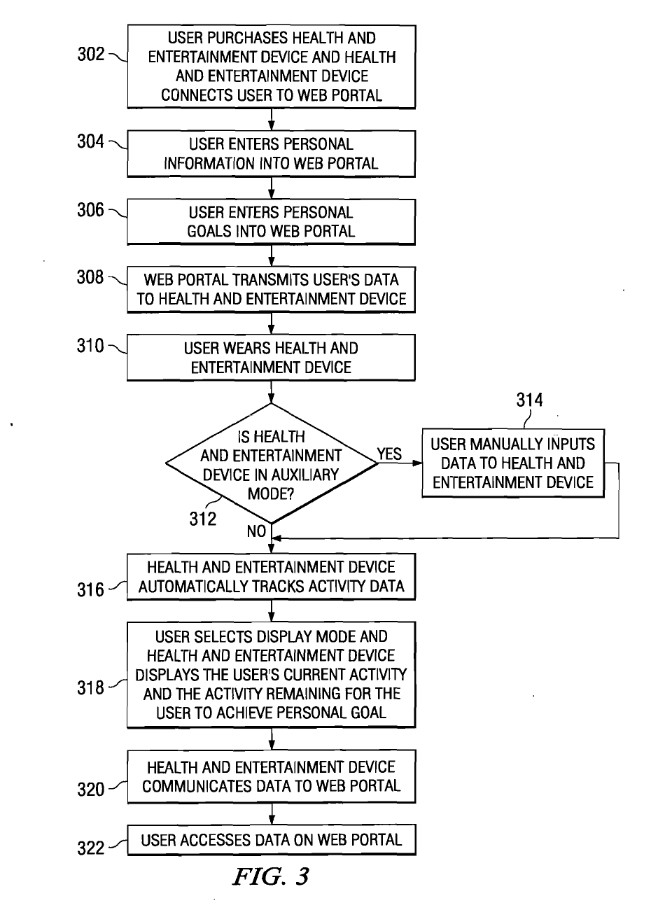

FIG. 3 is a simplified flowchart that illustrates an example method of the communication system 10 in accordance with an embodiment of the present invention. The flowchart begins at step 302, where user 12 purchases health and entertainment device 14 and connects health and entertainment device 14 to web portal 40. At step 304, user 12 inputs personal information into web portal 40 including user's height, weight, age, and sex. At step 306, user 12 also inputs personal goals into web portal 40. For example, user 12 may set one or more personal goals including eating three vegetables a day, expending four hundred calories per day, and spending five hours in the sport zone per week. At step 308, web portal 40 transmits user's data and goals to health and entertainment device 14. Goals can be color-coded on the device.

At step 310, user 12 wears health and entertainment device 14 by attaching clip 80 to an article of user's clothing. If health and entertainment device 14 is in auxiliary mode 58H during step 312, then the user 12 can manually input data into health and entertainment device 14 at step 314. For example, user 12 may press a button to increment the counter for the number of pills user 12 has taken for the day. In step 316, health and entertainment device 14 is constantly tracking user's 12 activity throughout the day while user 12 is wearing health and entertainment device 14. Health and entertainment device 14 is operable to dynamically update user's 12 personal goal information stored in health and entertainment device 14. For example, if user 12 has a daily goal of expending fifty calories and user has expended ten calories for the day, then the health and entertainment device 14 will update the goal such that user 12 only needs to expend thirty more calories to achieve his goal.

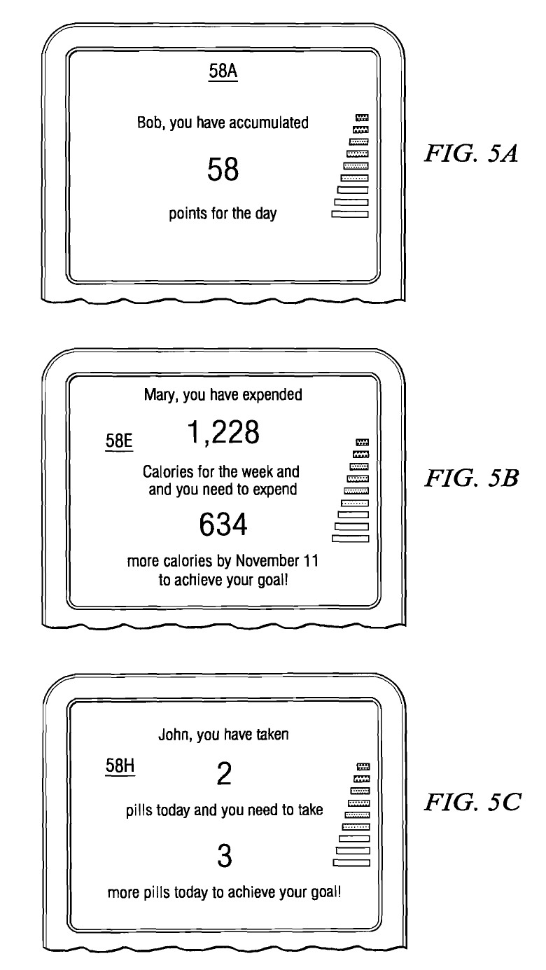

At step 318, user 12 can depress mode button 60 to toggle through the activity modes being displayed 58. If the activity mode displayed 58 does not have a goal associated with that activity, then health and entertainment device 14 displays the current activity data to user 12. An example of this display 58 is detailed below in FIG. 5A. If the activity mode displayed 58 does have a goal associated with that activity, then the health and entertainment device 14 displays the current activity and the activity remaining to achieve user's goal. An example of this display 58 is detailed below in FIG. 5B and FIG. 5C.

At step 320, the collected data may be transmitted to one or more computing devices 16 or web portal 40. This may be achieved in a wireless fashion, via a modem, a universal serial bus (USB) connection, or any other suitable connection, link, or port.

At step 322, the collected data may be accessed by any suitable entity authorized to do so. For example, user 12 himself may review the collected data via his home personal computer. In other scenarios, an employer may seek to review this collected data. In still other scenarios, a provider of healthcare may wish to ascertain this information. The collected data may be presented to these entities in any suitable format, which may be based on user preferences.

Note that the benefit of exercise has been well-documented. However, this descriptive information has not been quantified. For example, a study could suggest that the occurrence of a second heart attack could be reduced significantly if exercise was increased in the target population. But the bigger question would be: what level of activity and how much activity would solicit this positive response? Hence, activity monitor readings should be translated into some metric that provides a tool for ascertaining the dosage of exercise necessary to achieve a health objective. This would allow activity to be quantified for individuals and groups. Furthermore, this would allow the physician to prescribe a given dose of activity for a disease state such as coronary artery disease. For example, physician could prescribe the patient a dosage of activity, such as an exercise regimen that instructs the patient to use the activity monitor and walk thirty minutes a day in the health zone for five days a week.

The patient would know if he had achieved the prescribed dosage of activity from the data displayed on health and entertainment device 14 and the physician would be able to monitor the compliance of the patient by viewing the performance of the patient on the web portal.

Activity zones allow the physician and patient to know the amount of energy expended, energy intensity and time that was spent in a given zone. Hence, the activity component and the health benefits of various states can be accurately quantified. This would offer a powerful tool in evaluating, monitoring, quantifying, and managing the effects of activity of a given individual and his disease state.

In addition, an overall activity level of an individual or a group could be readily determined. This would allow for a correlation of health care costs (i.e. associated medical costs) and activity levels for various groups. Also, productivity levels could be correlated to activity levels identified through health and entertainment device 14. The measurements of health and entertainment device 14 could be used to provide a verifiable metric for comparing any number of various characteristics amongst individuals or groups.

FIG. 4 is a simplified flowchart that illustrates an example method of the special event feature of the health and entertainment device 14 in accordance with an embodiment of the present invention. At step 402, user 12 wears health and entertainment device 14 by attaching clip 80 to an article of user's 12 clothing.

If user 12 is not interacting with machines operable to communicate with health and entertainment device 14 during step 404, then user, 12 can manually input for special event 58I to begin at step 406. For example, user 12 can depress special event button 62 before user 12 runs a marathon. At step 408, user 12 engages in special event like running on a marathon. Health and entertainment device 14 measures all activity during the special event 58I. At step 410, user 12 can manually input for the special event 58I to end. For example, user 12 can depress special event button 62 again to end the special event when user 12 crosses the finish line at a marathon. At step 418, the health and entertainment device 14 displays the activity data measured during the special event 58I.

If user 12 is interacting with machines operable to communicate with health and entertainment device 14 during step 404, then special event mode 58I can automatically begin by a signal from the machine at step 412. For example, a treadmill may send a signal to health and entertainment device 14 to begin special event 58I when the treadmill is turned on. At step 414, user 12 engages in special event 58I like running on a treadmill. Health and entertainment device 14 measures all activity during the special event 58I. At step 416, machine can send a signal to health and entertainment device 14 to automatically end special event 58I. For example, a treadmill may send a signal to health and entertainment device 14 to end a special event 58I when the treadmill is turned off. At step 418, the health and entertainment device 14 displays the activity data measured during the special event 58I. Any of the special event data can be transmitted from health and entertainment device 14 to computing device 16, web portal 40, or server 32.

It is important to note that the stages and steps described above illustrate only some of the possible scenarios that may be executed by, or within, the present system. Some of these stages and/or steps may be deleted or removed where appropriate, or these stages and/or steps may be modified, enhanced, or changed considerably without departing from the scope of the present invention. In addition, a number of these operations have been described as being executed concurrently with, or in parallel to, one or more additional operations. However, the timing of these operations may be altered. The preceding example flows have been offered for purposes of teaching and discussion. Substantial flexibility is provided by the tendered architecture in that any suitable arrangements, chronologies, configurations, and timing mechanisms may be provided without departing from the broad scope of the present invention. Accordingly, communications capabilities, data processing features and elements, suitable infrastructure, and any other appropriate software, hardware, or data storage objects may be included within communication system 10 to effectuate the tasks and operations of the elements and activities associated with executing compatibility functions.

FIG. 5A is an example display 58 of user's daily points 58A, first introduced in FIG. 2, in accordance with an embodiment of the present invention. Health and entertainment device 14 can display customized messages because web portal 40 has transmitted user's personal data to health and entertainment device 14. In this example, user 12 does not have a goal associated with daily points 58A so only the current daily points 58A are displayed.

FIG. 5B is an example display 58 of user's goal for total weekly calories expended 58E, first introduced in FIG. 2, in accordance with an embodiment of the present invention. Health and entertainment device 14 can display customized messages because web portal 40 has transmitted user's personal data to health and entertainment device 14. In this example, user 12 has a goal associated with calories expended for the week 58E. As a result, both the current weekly calories expended 58E and the remaining weekly calories to be expended 58E to achieve user's goal are displayed.

FIG. 5C is an example display 58 of user's goal for pills consumed in auxiliary mode 58H, first introduced in FIG. 2B, in accordance with an embodiment of the present invention. Health and entertainment device 14 can display customized messages because web portal 40 has transmitted user's personal data to health and entertainment device 14. In this example, user 12 can have a goal, such as a nutritional regimen or pill regimen displayed in auxiliary mode 58H. As a result, both the current daily pills taken and the remaining daily pills to be taken to achieve user's goal or diet regimen are displayed.

Although the present invention has been described in detail with reference to particular embodiments, it should be understood that various other changes, substitutions, and alterations may be made hereto without departing from the spirit and scope of the present invention. The illustrated network architecture of FIG. 1A has only been offered for purposes of example and teaching. Suitable alternatives and substitutions are envisioned and contemplated by the present invention: such alternatives and substitutions being clearly within the broad scope of communication system 10. For example, the use of the LAN could easily be replaced by a virtual private network (VPN), a metropolitan area network (MAN), a wide area network (WAN), a wireless LAN (WLAN), or any other element that facilitates data propagation. Using analogous reasoning, the computer device illustrated by FIG. 1A may be supplanted by docking stations, gaming consoles, or any other suitable devices that are conducive to network communications. Furthermore, the health and entertainment device is not confined to displaying only the modes shown in FIG. 2B. In addition, any of the dimensions of device 14 can be changed or modified without parting from the essence of the present inventions. The dimensions illustrated and described offer just one of a multitude of possibilities.

Although the present invention has been described with several embodiments, a myriad of changes, variations, alterations, transformations, and modifications may be suggested to one skilled in the art, and it is intended that the present invention encompass such changes, variations, alterations, transformations, and modifications as fall within the scope of the appended claims.

1. A method for integrating and managing data, comprising:
interfacing with a user via a health and entertainment device, the device including:
an accelerometer, wherein the accelerometer is operable to track activity data by the user;
a music module operable to play music for the user;
a memory, wherein the memory stores data manually inputted by the user, and data received from the accelerometer; and
a processor, wherein the processor is operable to convert data from the accelerometer into one or more metrics based on data received from the memory, the health and entertainment device communicating with an athletic machine to begin a special event and to end the special event for the user while the health and entertainment device collects the activity data of the user during the special event, and wherein the machine is a selected one of a group of machines, the group consisting of:
a) a treadmill;
b) a stationary bicycle;
c) an elliptical machine; or
d) a stair master.

2. The method of claim 1, wherein the health and entertainment device includes a games module that allows the user to play games on the device, and to compete with other users in games provided on the games module.

3. The method of claim 1, wherein the health and entertainment device includes audio feedback for the user.

4. The apparatus of claim 1, wherein the health and entertainment device is operable to fit into one or more locations of smart clothing that includes fabricated receptacles for receiving the device.

5. The method of claim 1, further comprising:
providing a web portal located on one or more servers, the web portal operable to interact with the health and entertainment device, one or more servers coupled to a communication network providing access to the World Wide Web.

6. The method of claim 1, wherein the health and entertainment device further comprises:
a display;
one or more input buttons; and
a mode button, wherein the mode button selects a mode to be displayed, the mode being a selected mode of a group of modes, the group consisting of:
daily points accumulated;
average daily points accumulated for a week;
activity zone minutes;
daily calories expended;
total weekly calories expended;
daily distance traveled;
total weekly distance traveled;
an auxiliary mode, wherein the user manually inputs data;
a special event mode; and
a clock.

7. The method of claim 1, wherein the health and entertainment device automatically logs the user into a web portal.

8. The method of claim 7, wherein the web portal is operable to receive information relating to a user's age, height, weight, and sex, and wherein the web portal allows the user to create one or more goals for one or more modes.

9. The method of claim 7, wherein the web portal is further operable to transmit the user's age, height, weight, sex, and goals to the health and entertainment device.

10. The method of claim 9, wherein the health and entertainment device is further operable to track the user's one or more goals and to selectively display the user's one or more goals.

11. The method of claim 7, wherein the web portal is operable to monitor the user's data received from the health and entertainment device, and wherein the web portal is further operable to monitor a population's data received from the health and entertainment device, wherein the population is one or more users that communicate through the web portal.

12. (canceled)

13. (canceled)

14. The method of claim 1, wherein the health and entertainment device further comprises a skin, wherein the skin is replaceable with one or more skins, and wherein the one or more skins are a different color, material, and texture.

15. An apparatus for integrating and managing data, comprising:
a health and entertainment device that includes:
an accelerometer, wherein the accelerometer is operable to track activity data by a user;
a music module operable to play music while the user is active;
a memory, wherein the memory stores data manually inputted by the user, data received from the accelerometer, data received from one or more computer devices, and data received from the web portal; and
a processor, wherein the processor is operable to convert data from the accelerometer into one or more metrics based on data received from the memory, wherein the health and entertainment device includes audio feedback for the user, the audio feedback being triggered by activity levels of the end user, whereby the audio feedback is included in a group of audio feedback messages, the group consisting of an audio praise message if the user's activity has increased to a higher activity level, an audio warning message if the user's activity has increased to an unacceptable activity level, and an audio encouragement message if the user's activity has decreased to a lower level activity level.

16. The apparatus of claim 15, wherein the health and entertainment device includes a games module that allows the user to play games on the device and to compete with other users in games provided on the games module.

17. (canceled)

18. The apparatus of claim 15, wherein the health and entertainment device is operable to fit into one or more locations of smart clothing that includes fabricated receptacles for receiving the device.

19. The apparatus of claim 15, wherein the health and entertainment device further comprises a display operable to illustrate real-time data for the user.

20. The apparatus of claim 15, wherein the health and entertainment device is further operable to communicate with an athletic machine, wherein the machine is operable to begin and to end the special event mode.

21. The apparatus of claim 15, wherein the health and entertainment device further comprises a skin, wherein the skin is replaceable with one or more skins, and wherein the one or more skins are a different color, material, and texture.
